(19) United States
(12) Patent Application Publication
(10) Pub. No.: US 2007/0116757 A1
(43) Pub. Date: May 24, 2007

(54) Methylene Blue Derivatives

(75) Inventors: Roman V. Rariy, Allston, MA (US); Jane C. Hirsh, Wellesley, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Collegium Pharmaceutical, Inc.

(21) Appl. No.: 11/557,889

(22) Filed: Nov. 8, 2006

Related U.S. Application Data
(60) Provisional application No. 60/734,582, filed on Nov. 8, 2005.
(60) Provisional application No. 60/784,182, filed on Mar. 21, 2006.

(51) Int. Cl. A61K 31/5415; C07D 279/24; A61K 9/64; A61K 9/20

(52) U.S. Cl. 424/456; 424/464; 514/224.8; 544/41

(57) ABSTRACT

Pharmaceutical compositions comprising a fatty acid salt, a dicarboxylic acid salt, an alkyl sulfate salt, an aryl sulfate salt or an alkyl aryl sulfonate salt of methylene blue or a derivative of methylene blue are described herein. The compositions are preferably administered orally and can be administered as tablets, soft or hard shell capsules (e.g. soft gelatin capsules), suspensions or solutions. The composition can also be formulated as a suppository or enema or rectal administration. The compositions further comprise a pharmaceutically acceptable carrier and optionally one or more pharmaceutically acceptable excipients. Suitable excipients include diluents, binders, plasticizers, lubricants, disintegrants, colorants, stabilizers, surfactants, and combinations thereof. The fatty acid salts, alkyl sulate salts, aryl sulfate salts or alkyl aryl sulfonate salts can be co-mixed or co-melted with one or more fatty acids to make more hydrophobic compositions, which may result in less staining formulations. The compositions can be formulated for immediate release, controlled release such as extended release, delayed release, and pulsatile release, or combinations thereof. In one embodiment, the derivative of methylene blue is methylene dodecylsulfate.

### CROSS REFERENCE TO RELATED APPLICATIONS

This application claims priority under 35 U.S.C. §119 to U.S. Ser. No. 60/734,582filed Nov. 8, 2005 and U.S. Ser. No. 601/784,182 filed Mar. 21, 20066.

### FIELD OF THE INVENTION

This invention ls generally in the area of pharmaceutical compositions containing thiazine dyes, which have been modified for ease of handling, improved formulation capability, and/or modified release.

### BACKGROUND OF THE INVENTION

Dibenzo-1,4-thiazines, also known as phenothiazines, are a class of thiazine dyes which contain a six-membered heterocycle containing a single nitrogen atom and a single sulfur atom in which two benzene rings are fused to the heterocycle. Phenothiazine was first reported by Bernthsen in 1883 (Bernthsen, Ber. Deut. Chem. Ges., 16, 2896-2904, (1883)).

Methylene blue l; IUPAC name 3,7-bis(dimethylamino)phenothiazin-5-ium chloride) is the most well know example of the phenothiazine dyes. The structure of methylene blue is shown below. 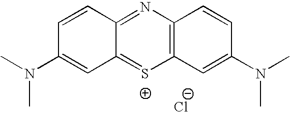
Methylene blue is used in a variety of applications such as textiles (for dyeing cellulosic fibers and printing leather), as an anti-oxidant and antiseptic, and in photogalvanic cells based on redox systems. Methylene blue and its analogues have also been used extensively for staining live and fixed tissues as well as for the diagnosis and treatment of disease (Anmerican Hospital Formulary Service Drug Information 2005). Derivatives such Azure A, B, and C as well as Taylor's Blue and Tolouidine blue are important dyes for the induction of metachromasia which is the ability of dyes to color different tissue constituents in different colors (Moura et al., Curr. Drug. Targ., 4, 133-141 (2003)). More recently Methylene blue has been studied for the treatment of blood products to inactive human immunodeficiency virus (“HIV”) and chronic hepatitis C virus infection (HCV). This new indication for Methylene Blue is initially intended to serve those countries where HCV is prevalent and cost effective treatment is essential to adoption of therapy. The World Health Organization estimates approximately 3 percent of the world's population (approximately 170-200 million people) are infected with HCV. Thiazine dyes, however, are highly staining materials which color the equipment used in the synthesis of the active and preparation of the dye-containing pharmaceutical compositions as well as the skin and clothing of those handling these compositions. Since methylene blue is a highly staining material, pharmaceutical manufacturers are generally unwilling to manufacture the dosage form. There exists a need for methylene blue pharmaceutical compositions which are effective in the diagnosis and/or treatment of disease but which are less staining to operators and facilities given the inherent problem with handeling this dye which is easily airborne and very sensitive to humidity (water).

Therefore, it is an object of the present invention to provide methylene blue formulations which are less staining and methods of making thereof.

It is further an on object of the invention to provide methylene blue formulations which are useful for treating or preventing viral infections and inactivating virus in blood and other biological fluid products.

It is a further object of this invention to provide derivatives of methylene blue that provide extended release of methylene blue.

### BRIEF SUMMARY OF THE INVENTION

Pharmaceutical compositions comprising a fatty acid salt, a dicarboxylic acid salt a long chain alkyl sulfate salt, an aryl sulfate salt or an alkyl aryl sulfonate salt of methylene blue or a derivative of methylene blue are described herein. In a preferred embodiment, the salt of methylene blue is methylene blue dodecysulfate. A particular benefit of the modified methlylene blue, and in particular, methylene blue dodecylsulfate, is that dye particles provide sustained release of the dye, unlike unmodified dye, which is highly soluble and dissolves immediately, resulting in rapid uptake and clearance following administration to an individual in need thereof. The rate of sustained release can be adjusted by varying the particle size. The compositions are preferably administered orally and can be administered in a variety of dosage forms including, but not limited to, tablets, soft gelatin capsules, hard shell capsules, suspensions, solutions, and emulsions. The compositions can also be formulated as a suppository or enema for rectal administration. The compositions can be formulated for immediate release, controlled release such as extended release, delayed release, and pulsatile release, or combinations thereof. These compositions typically include a pharmaceutically acceptable carrier and optionally one or more pharmaceutically acceptable excipients. Suitable excipients include diluents, binders, plasticizers, lubricants, disintegrants, colorants, stabilizers, surfactants, and combinations thereof. In one embodiment, fatty acid salts, dicarboxylic acid salts, alkyl sulfate salts, aryl sulfate salts and/or alkyl aryl sulfonate salts can be co-mixed or co-melted with one or more fatty acids to make more hydrophobic compositions, which may result in less staining formulations. In a preferred embodiment, methylene blue dodecylsulfate is co-melted with stearic acid and spray congealed to form beads. The beads can be encapsulated in an oral dosage form, such as a hard sheil capsule. in another embodiment the methylene blue dodecylsulfate particles are suspended in an excipient and loaded into soft gelatin capsules.

### BRIEF DESCRIPTION OF THE DRAWINGS

FIG. 1 shows the rate of release of methylene blue dodecylsulfate (expressed as methylene blue chloride trihydrate equivalents) versus time (hours) under physiological conditions.

### DETAILED DESCRIPTION OF THE INVENTION

I. Definitions

“Controlled release dosage form”, as used herein, refers to a dosage form for which the drug release chracteristics of time course and/or Iocation are chosen to accomplish therapeutic or convenience objectives not offered by conventional immediate release dosage forms such as solutions or promptly dissolving dosage forms. Delayed release, extended release, and pulsatile release formns and their combinations are types of controlled release dosage forms.

“Delayed release dosage form”, as used herein, refers to a dosage form that releases a drug (or drugs) at a time other than promptly after administration.

“Extended release dosage form”, as used herein, refers to a dosage form that allows at least a twofold reduction in dosing frequency as compared to that drug presented as a conventional dosage form (e.g. as a solution or prompt drug-releasing, conventional solid dosage form).

“Pulsatile release dosage form”, as used herein, refers to a dosage form that mimics a multiple dosing profile without repeated dosing and allows at least a twofold reduction in dosing frequency as compared to that drug presented as a conventional dosage form. A pulsatile release profile is characterized by a time period of no release (lag time) followed by rapid drug release.

“Soft capsule”, as used herein, refers to a one-piece, hermetically sealed soft shell capsule containing a liquid, a suspension, or a semi-solid fill material. Soft capsule shells can be prepared from gelatin or non-gelatin materials such as polysacharides. Capsules may consist of two pieces that have been juxtaposed.

II. Composition

A. Methlylene Blue and Its Derivatives

The compounds described herein are fatty, acid salts, dicarboxylic acid salts, long, chain alknyl suilfate salts, aryl sulfate salts, or alkyl aryl sulfotnate salts of compounds having the chemical fomula shown below: 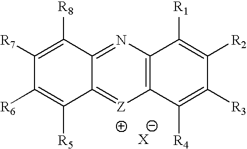
wherein R₁, R₂, R₄, R₅, R₇ and R₈ are independently selected from the group consisting of hydrogen; linear, branched or cyclic alkyl, linear, branched or cyclic substituted alkyl; aryl; substituted aryl alkoxy; thioalkoxy; alkylamino; nitro; amino; and halogen; R₃ and R₆ are independently selected from the group consisting of —OR₈, —NHR₉, and —NR₁₀R₁₁ wherein R₈-R₁₁ is a linear, branched or cyclic substituted or unsubstituted hydrocarbon or R₁₀ and R₁₁ together with the nitrogen atom to which they are attached form an optionally substituted 5-, 6-, or 7-membered ring; wherein X⁻ is a counterion and wherein Z is either S or O. Suitable counter-ions include, but are not limited to, fatty acids, dicarboxylic acids, long chain alkyl sulfates, aryl sulfates, or alkyl aryl sulfonates. Metabolites of the compounds described by the chemical formula above can also be used. For example, leucomethylene blue, the structure of which is shown below, is a metabolite of methylene blue,

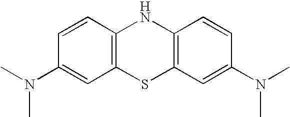

Examples of useful thiazine dyes include, but are not limited to, methylene blue methyl methylene blue, dimethyl methylene blue, azure A, azure B azure C, methylene green, new methylene blue, Taylor's Blue, Toluidine Blue O, and thionine. These dyes are all commercially available from a number of different sources. Symmetrical 3,7-bi(dialkylamino)phenothiazin-5-ium derivatives which may be useful are described in Moura et al., Current Drug Targets, Vol. 4, 133-141 (2003). Derivatives of methylene blue in which the methly groups of methylene blue have been replaced with ethyl, n-propyl, n-butyl, n-pentyl, and a n-hexyl groups are described in Mellish et al., Photochemistry and Photobiology, Vol. 75, No 4, pp, 392-397 (2002). Finally, phenoxazine dyes, in which the sulfur atom of the thiazine ring is replaced by an oxygen atom, may also be used. Examples of phenoxazine dyes include Nile Blue and its derivatives.

Methylene blue, 3,7-Bis(dimethylamino)-phenothiazin-5-ium chloride, C₁₆H₁₈ClN₃S, is a dark green or blue thiazine dye which was first isolated in 1876. The dye is soluble in water and sparingly soluble in alcohol, forming deep blue solutions.

Suitable fatty acids which can be used to prepare the salts include, but are not limited to, butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic), acid, triacontanoic (melissic) acid, dotriacpntanoic (lacceroic) acid, tritriacontanoic (ceromelissic) acid, tetratriacontanoic (geddic) acid, and pentatriacontanoic (ceroplastic) acid. It is important to note that yield and efficacy of the synthesis of the different salt forms will vary anmong the various salts.

Dicarboxylic acids can also be used to prepare the salts of these compounds. Suitable dicarboxylic acids include, but are not limited to succinic, glutaric, adipic, pimelic, suberic, azelaic, sebacic, dodecanedioic, brassylic, thapsic, undecanedioic, tetradecanecdioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octenedioic, and trans-3-octenedioic acid.

Suitable alkylsulfates include, but are not limited to, sodium, potassium, and ammonium salts of long chain alkyl sulfates sucxh as decanoic (capric) sulfate, dodecyl (lauric) sulfate, tetradecanoyl (myristic) sulfate, hexadecanoyl (palmitic) sulfate, heptadecanoyl (margaric) sulfate, octadecanoyl (stearic) sulfate eicosanoyl (arachidic) sulfate, docosanoyl (behenic) sulfate, tetracosanoyl (lignoceric) sulfate, hexacosanoyl (cerotic) sulfate, heptacosanoyl (carboceric) sulfate, octacosanoyl (montanic) sulfate, t riacontanoyl (melissic) sulfate dotriacontanoyl (lacceroic) sulfate, tritriacontanoyl (ceromelissic) sulfate, tetratriacontanoyl (geddic) acid, and pentatriacontanoyl (ceroplastic) sulfate.

Suitable alkyl aryl sulfonate include, but are not limited to, sodium, potassium, and ammonium salts of alkyl aryl sulfonates such as dodecylbenzene sulfonate, tetradecanoylbenzene sulfonate, hexadecanoyl-benzene sulfonate, heptadecanoylbenzene sulfonate, octadecanoylbenzene sulfonate, eicosanoylbenzene sulfonate, docosanoylbenzene sulfonate, tetracosanoylbenzene sulfonate, hexacosanoylbenzene sulfonate, heptacosanoyl-benzene sulfonate, octacosanoylbenzene sulfonate, triacontanoylbenzene sulfonate, dotriacontanoylbenzene sulfonate, tritriacontanoylbenzene sulfonate, tetratriacontanoylbenze sulfonate, and petatriacontanoylbenzene sulfonate.

In a preferred embodiment, the salt is the dodecylsulfate salt of methylene blue or a derivative of methylene blue. The dodecylsulfate salt can be further co-mixed or co-melted with a fatty acid or a dicarboylic acid to make the composition more by hydrophobic and thus less prone to staining. In one embodiment, methylene blue dodecylsulfate is co-melted or co-mixed with stearic acid. Suitable fatty acids include, but are not limited to, butanoic (butyric) acid, petanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid tritriacontanoic (ceromelissic) acid, tetratriacontanoic (geddic) acid, and pentatriacontanoic (ceroplastic) acid. Suitable dicarboxylic acid include, but are not limited to, succinic, glutaric, adipic, pimelic, suberic, azelaic, sebacic, dodecanedioic, brassylic, thapsic, undecanedioic, tetradecanedioic, pentadecanedioic, hexacdecanedioic, octadecanedioic, traumatic acid, itaconic (methylenesuccinic), trans-2-hexenedioic, trans-3-hexenedioic, cis-3-ocetenedioic, cis-4-octenedioic, and trans-3-octenedioic acid.

A particular benefit of the modified methylene blue, and in particular, methylene blue dodecylsulfate, is that dye particles provide sustained release of the dye, unlike unmodified dye, which is highly soluble and dissolves immediately, resulting in rapid uptake and clearance following administration to an individual in need thereof. The rate of sustained release can be adjusted by varying the particle size.

B. Additional Active Ingredients

The dye formulation may also be administered in combination with one or more other active agents such as analgesics, antibiotics, antifungals, antivirals, anti-inflammatory drugs, antipyretics, nutritional agents, vitamins, and parasympathomimetics, or one or more vitamins such as vitamins C, E, and B-complex vitamins.

C. Additive, Excipients and Carriers

The compounds can be administered as tablets, hard or soft shell capsules (e.g. soft gelatin capsules), suspension, solutions, or emulsions, or suppositories.

In one embodiment, the dodecylsulfate salt of methylene blue or a derivative of methylene blue is added into an oil and/or a suspension in which methylene blue dodecylsulfate particles are insoluble and filled into soft gelatin capsules.

Formulations may be prepared using a pharaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders lubricants, disintegrators, fillers, solubilizing agents, pH modifying agents, preservatives, stabilizers, such as anti-oxidants, wetting or emulsifying agents, suspending agents and coating compositions. “Carrier” also includes all components of any coating composition, which may include plasticizers, pigments, colorants, stabilizing agents, glidants, pore formers and surfactants.

Diluents, also referred to as “fillers,” are typically necessary to increase the bulk of a solid dosage form so that a practical size is provided for compression of tablets or formation of heads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a table or bead or granule remains intact after the formation of the dosage forms. Suitable binder materials include, but are not limited to, starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubircants are used to facilitate tablet manufacture. Examples of suitable lubricants include, but are not limited to, magnesium stearate, calcium stearate, stearic acid glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

III. Methods of Manufacturing

A. Fatty Acid Salts of Methylene Blue

Methylene blue hydrochloride and a salt of the desired fatty acid may be dissolved in an appropriate solvent, such as methylene chloride or chloroform, and heated to reflux to facilitate an ion exchanmge. Methylene blue hydrocloride can also be mixed with a salt of the desired fatty acid in an aqueous environment to form an organic solvent soluble ion pair, which can be extracted into organic solvents, such as methylene chloride or chloroform.

Alkyl Sulfate Salts of Methylene Blue

Alkyl sulfate salts of methylene blue can be prepared in a number of ways. For example, methylene blue and a metal alkyl sulfate, such as sodium dodecylsulfate, are dissolved in water and heated to reflux in the presence of water-immiscible organic solvent. The organic phase is separated, washed, dried, filtered, and concentrated to give methylene blue alkylsulfate. It is expected that aryl sulfate and alkyl aryl sulfonate salts can be prepared in a similar manner.

C. Controlled Release Formulations

As will be appreciated by those skilled in the art and as described in the pertinent texts and literature, a number of methods are available for preparing drug-containing tablets, beads, granules or particles that provide a variety of drug release profiles. Such methods include, but are not limited to, the following coating a drug or drug-containing composition with an appropriate coating material, typically although not necessarily incorporating a polymeric material, increasing drug particle size placing the drug within a matrix, and forming complexes of the drug with a suitable complexing agent.

Delayed release dosage formulations may be prepared as described in standard refeerences such as “Pharmaceutical dosage form tablets”, eds. Liberman et al. (New York, Marcel Dekker, Inc., 1989), “Remington—The science and practice of pharmacy”, 20th ed. Lippincott Williams & Wilkins, Baltime, Md., 2000, and “Pharmaceutical dosage forms and drug delivery systems”, 6^{th} Edition, Ansel et al., (Media, Pa.: Williams and Wilkins, 1995). These references provide information on carriers, materials, equipment and process for preparing tablets, soft gelatin capsules, hard shell capsules, and delayed release dosage forms of tablets, capsules, and granules.

### Extended Release Formulations

Extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug with a slowly dissolving or slowly swelling polymer carrier into a tablet form. The three major types of materials used in the preparation of matrix, devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and CARBOPOLE® 934, polyethylene oxides. Suitable fats and fatty compounds include fatty alcohols (such as lauryl, myristyl stearyl, cetyl or cetostearyl alochol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name STEROTEX®, stearic acid, cocoa butter, and stearyl alcohol. Suitable waxes and was-like materials include natural or synethetic waxes, hydrocarbons, and normal waxes. Specific examples of waxes include beeswax, glycowax, castor wax, carnauba wax, parallins and candelilla wax. As used herein, a wax-like material is defined as any material which is normally solid at room temperature and has a melting point of from about 30 to 300° C.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsuls containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceuticl ingredient. The usual diluents include inert powdered substances such as any of many different kins of starch, powdered cellulose, especially crystalline and microcrystalline cellulose, sugars such as fructose, mannitol and sucrose grain flours and similar edible powders. Typical diluents include, for example, various types of starch, lactose, mannitol, kaolin, calcium phosphate or sulfate, inorganic salts such as sodium chloride and powdsered sugar. Powdered cellulose derivatives are also useful. Typical table binders include substances such as starch, gelatin and sugars such as lacrose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubranct is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an agueous dispersion met hod. In a congealing method, the drug is mixed with a wax material and either spray-congealed or congealed and screened and processed to form beads. In one embodiment, methylene blue dodecylsulfate is dissolved in molten stearic acid and the mixture is spray congealed to form beads. The beads can be encapsulated in a dosage form, such as a hard gelatic capsule. The release rate of the active agent can be varied by varying the size of the beads. The release rate can also be modified by incorporating one or more materials which loosen up the matrix and allow the dissolution medium to interact with the active agent. Suitable materials include, but are not limited to, other waxy materials, plasticizers, hydrophilic materials including, but not limited to, polyethylene glycols.

A preferred method for preparing extended release tables is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide plasticizers or the like. The admixture is used to coat a bead core such as a sugar spheere (or so-called “non-pareil”) having a size of approximately 60 to 20 mesh.

### Delayed Release Formulations

Delayed release formulations are created by coating a solid dosage form with a film of a polymer which is insoluble in the acid environment of the stomach, and soluble in the neutral environment of small intestines.

The delayed release dosage units can be preparfed, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a table or capsule. Preferred coating materials include bioerodible, gradually hydrolyzable, gradually water-soluble, and/or enzymaticall degradable polymers, and may be conventional “enteric” polymeers. Enteric polymers, as will be appreciated by those skilled in the art. become soluble in the higher pH environment of the upper gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethy cellulose cellulose acetate, cellulose acetate phthalate, cellulose acetate trimelitate and carboxymethylcellulose sodium, acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma, Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITs®. NE, RL, and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer, enzymatically degradable polymers such as azo polymers, pectin, chitosan, amylose and guar gum, zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colerants, stabilizing agents, glidants, etc. A plasticizer is normally present to reduce the fragility of the coating, and will generally represent about 11 wt. % to 50 wt. % relative to the dry weight of the polymer. Examples of typical plasticizers include polyethylene glycol, propylene glycol, triacetin, dimethyl phthalate, diethyl phthalate, dibutyl phthalate dibutyl sebacate, triethyl citrate, tributyl citrate, tiethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommenmed to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage forms, see Pharmaceutical Dosage Forms: Tablets, eds. Lieberman et al. (New York;: Marcel Dekker, Inc., 1989), and Ansel et al., Pharmaceutical Dosage Forms and DSrug Delivery Systems, 6^{th} Ed. (Media, Pa.: Williams & Wilkins, 1995).

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Alternatively, the drug can be continuously delivered to a patient over an extended period of time using a controlled release polymeric implant. Polymeric implants are generally manufactured from polymers which degrade in vivo over a known period of time. Examples of useful polymers include polyanhydrides, polylactic acid, polyothoester, and ethylene vinyl acetate.

Capsules

i. Soft Shell Capsules

Soft capsules ar composed of a capsule content (“fill”) encapsulated in a soft gelatin or non-gelatin shell. Non-gelating materials include carbohydrates such as carrageenan and starches. For soft capsules manufactured using a rotary die encapsulation process, the fill is typically a liquid or a combination of miscible liquids, a solution of a solid(s) in a liquid(s), or a suspension of solid(s) in a liquid. The capsule shell is composed primarily of gelatin or non-gelatin materials, a plasticizer, and purified watr. In addition to the plasticizer(s), other suitable shell additives include opacifiers, colorants, humectants, preservatives, flavoring, and buffering salts and acid.

The ingredients are combined to form a molten gelatin mass using either a cold melt or a hot melt process. The prepared gel masses are transferred to preheated, temperature-controlled, jacketed holding tanks when the gel mass is aged at 50-60° C. until used for encapsulation. Soft capsules are typically produced using a rotary die encapsulation process. The gel mass is fed either by gravity or through positive displacement pumping to two heated (48-65° C.) metering devices. The metering devices control the flow of gel into cooled (10-18° C.), rotating casing drums. Ribbons are formed as the cast gel masses set on contact with the surface of the drums.

The ribbons are fed through a series of guide rolls and between injection wedges and the capsule-forming dies. A food-grad lubricant oil is applied to the ribbons to reduce their tackiness and facilitate their transfer. Suitable lubricants include mineral oil, medium chain triglycerides, and soybean oil. Fill formulations are fed into the encapsulation machine by gravity.

In one embodiment, methylene blue dodecylsulfate particles are suspended in any oil in which the particles are not soluble and encapsulated into the soft gelatin capsule.

In one embodiment, methylene blue hydrochloride is dissolved in the gelatin capsule shell and dodecylsulfate salt of methylene blue or a dodecylsulfate salt of a derivative of methylene blue is encapsulated in the gelatin capsule shell. The methylene blue hydrochloride incorporated into the capsule shell can provide an immediate release dose. The soft gelatin capsule can then be coated with a non-aqueous coat to propect it from moisture. The dodecylsulfate salt of methylene blue or dodecylsulfate salt of a derivative of methylene blue, which is encapsulated within the gelatin capsule shell, can be formulated to controlled release (e.g. delayed release, extended release, pulsatile release or combinations thereof). The shell and/or the fill material can further comprise one or more pharmaceutically acceptable excipients. Soft gelatin capsules are described in “Liquid Filled and Sealed Hard Gelatin Capsules” by Ewart T. Cole of Capsugel.

ii. Hard Shell Capsules

Hard shell capsules differ from soft gel capsules primarily in the amount of plasticizer present in the capsule shell. Hard shell capsule contain little or no plasticizer, while soft shell capsules contain a plasticizer, such as glycerin, in an amount up to about 30% by weight of the capsule shell. Generally, the moisture uptake of soft gelatin capsules plasticized with glycerol is considerably higher than that for hard gelating capsules. In addition, the permeability of the capsule shell is generally lower for hard shell capsules than for soft shell capsules due to the presence of plasticizer in the soft shell capsule.

Another difference between hard and soft capsules is the encapsulation processes. In the hard gelatin capsule process, the capsule is pre-fabricated and supplied empty, whereas in the soft gelatin capsule process the encapsulation and filling take place simultaneously. The moisture content of the gelatin/plasticizer mass at this stage can be around 50%, the equilibrium moisture level only being reached after several days storage on trays.

In one embodiment, a mixture of methylene blue dodecylsulfate and stearic acid is prepared by dissolving the materials in an approprioate solvent system or by co-melting the materials. The mixture is then spray congealed to form particles, or beads, which are encapsulated in a hardshell capsule.

E. Suppositories

Suppositories are solid dosage forms intended for administration of drugs via the rectum, vagina or urethra that melt, soften or dissolve in the body cavity. The drug is incorporated into a base such as cocoa butter which melts at body temperature, or into a base such as glycerinated gelatin or polyethylene glycol (PEG) which slowly dissolves inthe mucous secretions. Suppositories are suited particularly for producing local action, but may also be used to produce a systemic effect. Suppositories can be prepared, on an industrial scale, by compression molding or fusion molding.

Compression molding is a method of preparing suppositores from a mixed mass of grated suppository base and medicaments which is forced into a special compression mold. The method requires that the capacity of the molds first be determined by compressing a small amount of the base into the dies and weighing the finished suppositories. When active ingredients are added, it is necessary to omit a portion of the suppository base, based on the density factors of the active ingredients.

Fusion Molding involves first melting the suppository base, and then dispensing or dissolving the druge in the melted base. The mixture is removed from the heat and poured into a suppository mold. When the mixture has congealed, the suppositories are removed from the mold. The fusion method can be used with all types of suppositories.

IV. Method of Treatment

The preferred dosage range for methylene blue or its derivative for treating or preventing a viral infection such as Hepatitis C or human immunodeficiency virus is 30 to 180 mg twice a day, more preferably between 60 and 130 mg twice a day, or a dosage which yields blood levels between 0.2 and 2000 μM and more preferably less than 200 μM. The drug is preferably administered enterally although it can also be administered parenterally. The method described herein does not require admimistration of exogenous light, although the results may be enhanced by exposure to light in addition to that normally transmitted through the skin. Exposure to light can occur with exposure to sun light, a tanning light, or even incandescent light. The thiazine dyes can also be delivered using techniques known to those skilled in the art of drug delivery to target specific cell types or to enhance the activity of the dye.

Unless defined otherwise, all technical and scxientific terms used herein have the same meeansing as commonly understood by one of skill in the art to which the disclosed invention belongs. The teaching of the references cited herein are specifically incorporated by reference.

Those skilled in the art will recognize, or be able to ascertain using no more that routine experimentation, many equivalents to the specific embodiments of the invention described herein. Such equivalents are intended to be encompassed by the following claims.

### EXAMPLES

### Example 1

### Synthesis of the Dodecylsulfate Salt of Methylene Blue

27.77 g (86.8 mmol) of methylene blue chloride and 50 g (173.3 mmol) of sodium dodecylsulfate were heated at reflux for 24 h in 1.7 L of CH₂Cl₂ and 250 ml of water. The layers were seprated, and the organic phase was washed with wat er (3×200 ml), and dried over sodium sulfate. Filtration and concentration gave 18.89 g of the dodecylsulfate salt of methylene blue. The structure of methylene blue dodecylsulfate was confirmed by ¹H NMR and mass spectrometry. Elemental analysis showed that chlorine was not present in the product, indicating that the product was free of methylene blue chloride.

### Example 2

### Preparation of Co-Melts of Methylene Blue Dodecyl Sulfate and Stearic Acid

Stearic acid was placed in a scintillation vial and the stearic acid was melted in an oil bath at 95° C. Methylene blue dodecylsulfate (MBDS) was added to the molten stearic acid (SA) and mixed well until a homogenous mixture was obtained (approximately 10-15 minutes). The uniform melt as poured onto an aluminum foil tray and allowed to solidify, resulting in a thin layer of the mixture. No distinct MBDS particles were observed in the molten or solidified product when a 1:10 ratio of MBDS:SA was used. MEDS insoluble particles were observed when a 1:2 ratio of MBDS:SA was used. All solubility studies were conducted using a composition with a 1:10 ratio. The ratios werfe calculated based on the methylene blue chloride equivalent.

This study demonstrated that MEDS dissolves in molten stearic acid and remains incorporated in the stearic acid matrix upon solidifying, indicating that MBDS dissolved in stearic acid is useful as a wax-based extended release formulation.

### Example 3

### Solubility of Methylene Blue Dodecylsulfate and Methylene Blue Dodecylsulfate-Stearic Acid Co-melts

Known amounts of methylene blue chloride (MBC), methylene blue dodecylsulfate (MBDS), and methylene blue dodecylsulfate-stearic acid melt (MBDS-SA) were each placed in a scintillation vial. The solubility of the compound was evaluated by adding 4 ml of deionized water, phosphate buffer, or phosphate buffeer containing 0.75% Tween 20 and 0.15 M NaCl to the vials. The vials were sealed and shaken at 250 rpm using a bench top shaker at room temperature for at least 24 hours. The samples were centrifuged and the superantant was collected for analysis. Each vial contained some non-dissolved material indicating that a saturated solution had been obtain. The results are shown in Table 1.

TABLE 1

| | Solubility (mg/ml) given as Methylene Blue Chloride equivalent | | | | | | | | |
|---|---|---|---|---|---|---|---|---|---|
| | | | | | | | phosphate buffer pH 6.8 with 0.75% | | |
| | de-ionized water | | | phosphate buffer pH 6.8 | | | (w/w) Tween 20 and 0.15 M NaCl | | |
| Sample | sample 1 | sample 2 | Average | sample 1 | sample 2 | Average | sample 1 | sample 2 | Average |
| MBC | 34 | 38 | 36 | 37 | 38 | 38 | 29 | 28 | 29 |
| MBDS | 0.01 | 0.01 | 0.01 | 0.13 | 0.14 | 0.14 | 0.48 | 0.53 | 0.51 |
| MBDS-SA | 0.03 | 0.02 | 0.03 | 0.19 | 0.18 | 0.19 | 1.46 | 1.55 | 1.51 |
| (1:10) | | | | | | | | | |

Table 1 shows that MBDS and MBDS-SA melt are substantially less soluble than MBC in all solvent systems tested. This indicates that MBDS and MBDS-SA, as well as dosage forms containing these materials, are less staining than MBC. The low saturation solubilityof MBDS and MBDS-SA melt also suggersts that these compounds may exhibit sustained release properties.

### Example 4

### Dissolution of 40-60 Mesh MBDS Particles

40-60 mesh size particles (425-250 microns) of methylene blue dodecylsulfate (MBDS) were separated from the bulk MBDS material and their dissolution was evaluated (see Example 1 for method of manufacturing). Each disolution vessel contained 30 mg of 40-60 mesh size particles. The dissolution conditions were 0.1 N HCl for 1 hour (Acid stage) followed by phosphate buffer (pH 6.8) supplemented with 0.75% Tween 20 and 0.15 M NaCl (Buffer stage) at 37° C. A USP dissolution Apparatus II (paddles) was used at 50 rpm.

The samples were withdrawin, diluted two fold with phosphate buffer (pH 6.8 containing 100 mM SDS), and their absorbance was measured at 664 nm. The methylene blue concentration in the dissolution media was determined using calibration curves obtained with methylene blue chloride trihydrate in phosphate buffer (pH 6.8 supplemented with 50 mM SDS).

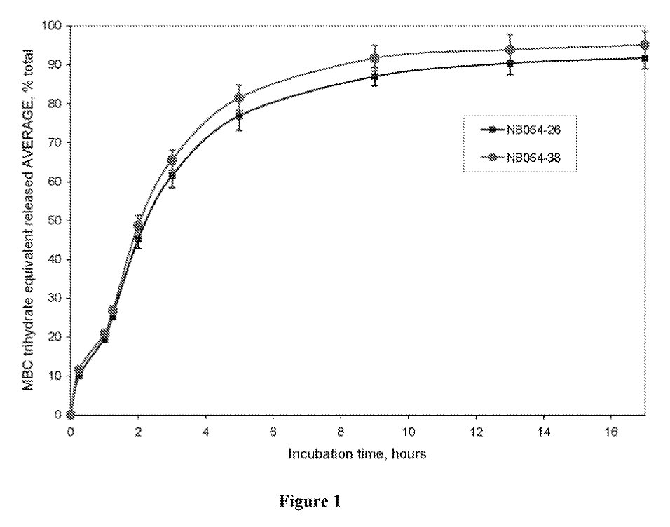

Two separate sets of experiments were conducted to confirm the reproducibility of the obtained results (NB064-26 and NB064-38). The percent average dissolution values as a function of time for two different lots of MBDS are shown in FIG. 1. The graph in FIG. 1 shows that the 40-60 mesh MBDS particles provide extended release of Methylene Blue for up to 8 hours. It is believed that the release curve can be altered by varying the particl size of MBDS since particle size determines total surface area and thus influences the rate of release.

We claim:

1. A pharmaceutical composition comprising a salt of methylene blue or a salt of a derivative of methylene blue selected from the group consisting of fatty acid salts, dicarboxylic acid salts, alkyl sulfate salts, aryl sulfate salts, alkyl aryl sulfonate salts and combinations thereof, in a pharmaceutically acceptable carrier.

2. The composition of claim 1, wherein the salt of methylene blue is an alkylsulfate salt.

3. The composition of claim 2, wherein the alkylsulfate salt of methylene blue is methylene blue dodecylsulfate.

4. The composition of claim 1 wherein the derivative of methylene blue has the chemical formula shown below: 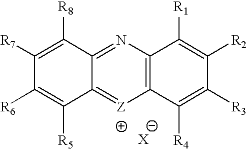
wherein R₁, R₂, R₄, R₅, R₇ and R₈ are independently selected from the group consisting of hydrogen, linear, branched or cyclic alkyl, aryl, substituted aryl, alkoxy, thioalkoxy, alkylamino, nitro, amino and halogen; R₃ and R₆ are independently selected from the group consisting of —OR₈, —NHR₉, and —NR₁₀R₁₁ and combinations thereof wherein R₈-R₁₁ is a linear, branched or cyclic hydrocarbon or R₁₀ and R₁₁ together with the nitrogen atom to which they are attached from an optionally substituted 5-, 6-, or 7-membered ring, wherein X⁻ is a counterion and wherein Z is either S or O and metabolites thereof.

5. The composition of claim 1 wherein the derivative of methylene blue is selected from the group consisting of methyl methylene blue, dimethyl methylene blue, azure A, azure B, azure C, methylene green, new methylene blue, Taylor's Blue, Toluidine Blue O, thionine, Nile blue, and metabolites thereof.

6. The composition of claim 1 further comprising at least one other active agent selected from the group consisting of analgesics, antibiotics, antifungals antivirals, anti-inflammatory drugs, antipyretic, nutritional agents, vitamins, and parasympathomimetics.

7. The composition of claim 6 comprising one or more vitamins selected from the group consisting of vitamins C, E, and B-complex.

8. The composition of calim 1 wherein the composition is in a dosage form selected from the group consisting of tablets, soft gelatin capsules, hard shell capsules, suspensions, solutions and suppositories.

9. The composition of claim 1 wheerein the composition is a controlled release formulation selected from the group consisting of immediate release, delayed release, extended release, pulsatile release, and combinations thereof.

10. The composition of claim 1 wherein the fatty acid used to form the salt of methylene blue or a derivative of methylene blue is selected from the group consisting of butanoic (butyric) acid pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (steaaric) acid, eicosanoic (arachidic) acid docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceromelissic) acid, tetratricontanoic (geddic) acid, and pentatriacontanoic (ceroplastic) acid.

11. The composition of claim 1 wherein the dicarboxylic acid used to form the salt of methylene blue or a derivative of methylene blue is a dicarboxylic acid selected from the group consisting of succinic, glutaric, adipic, pimelic, suberic, azelaic, sebacic, dodecanedioic, brassylic, thapsie, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, dis-4-octenedioic, and trans-3-octenedioic acid.

12. The composition of claim 1, wherein the alkyl sulfate used to form the salt of methylene blue or a derivative of methylene blue is selected from the group consisting of decanoic (capric) sulfate, dodecyl (lauric sulfate, tetradecanoyl (myristic) sulfate, hexadecanoyl (palmitic) sulfate, heptadecanoyl (margaric) sulfate, octadecanoyl (stearic) sulfate, eicosanoyl (arachidic) sulfate, decosanoyl (behenic) sulfate, tetracosanoyl (lignoceric) sulfate, hexacosanoyl (cerotic) sulfate, heptacosanoyl (carboceric) sulfate, octacosanoyl (montanic) sulfate, triacontanoyl (melissic) sulfate, dotriacontanoyl (lacceroic) sulfate, tritriacontanoyl (ceromelissic) sulfate, tetratriacontanoyl (geddic) sulfate, and pentatriacontanoyl (ceroplastic) sulfate.

13. The composition of claim 1, wherein the alkyl aryl sulfonate used to form the ssalt of methylene blue or a derivative of methylene blue is selected from the group consisting of dodecylbenzene sulfonate, tetradecanoylbenzene sulfonate, hexadecanoyl-benzene sulfonate, heptadecanoylbenzene sulfonate, octadecanoylbenzene sulfonate, eicosanoylbenzene sulfonate, docosanoylbenzene sulfonate, tetracosanoylbenzene sulfonate, hexacosanoylbenzene sulfonate, heptacosanoyl-benzene sulfonate, octacosanoylbenzene sulfonate, triacontanoylbenzene sulfonate, dotriacontanoylbenzene sulfoante, tritriacontanoylbenzene sulfonate, tetratriacontanoylbenzene sulfonate, and pentatriacontanoylbenzene sulfonate.

14. The composition of claim 1 further comprising a fatty acid selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

15. The composition of claim 1 further comprising a dicarboxylic acid selected from the group consisting of succinic glutaric, adipic, pimelic suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic cis-4-octenedioic, and trans-3-octenedioic acid.

16. The composition of claim 1 wherein the derivative of methylene blue is a metabolite of methylene blue which has the structure shown below:

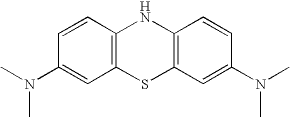

17. The composition of claim 8, wherein the dosage form is a soft shell capsule.

18. The composition of claim 17, further comprising methylene blue dissolved in the soft gelatin capsule shell to provide an immediate release portion.

19. A compound of formula I: 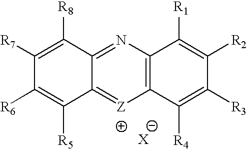
wherein R₁, R₂, R₄, R₅, R₇ and R₈ are independently selected from the group consisting of hydrogen, linear, branched or cyclic alkyl, aryl, substituted aryl, alkoxy, thioalkoxy, alkylamino, niotro, amino and halogen, R₃ and R₆ are independently selected from the group consist ing of —OR₈, —NHR₉, and —NR₁₀R₁₁ and combinations thereof wherein R₈-R₁₁ is a linear, branched or cyclic hydrocarbon or R₁₀ and R₁₁ together with the nitrogen atom to which they are attached form an optionally substituted 5-, 6-, or 7-membered ring, wherein Z is either S or O, and whrein X is a counterion derived from a compound selected from the group consisting of fatty acids, alkyl sulfates, aryl sulfates, and alkyl aryl sulfonates.

20. The compound of claim 19, wherein the fatty acid used to form the salt of methylene blue or a derivative of methylene blue is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, tricontanoic (melissic) acid, dotriacontanoic (lacceroic) acid, tritriacontanoic (ceromelissic) acid, tetratriacontanoic (geddic) acid, and petatriacontanoic (ceeroplastic) acid.

21. The compound of claim 19, wherein the dicarboxylic acid used to form the salt of methylene blue or a derivative of methylene blue is a dicarboxylic acid selected from the group consisting of succinic, glutaric, adipic, pimelic, suberic, azzelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octenedioic, and trans-3-octendioic acid.

22. The compound of claim 19, wherein the alkyl sulfate used to form the salt of methylene blue or a derivative of methylene blue is select ed from the group consisting of decanoic (capric) sulfate, dodecyl (lauric) sulfate, tetradecanoyl (myristic) sulfate, hexadecanoyl (palmitic) sulfate, heptadecanoyl (margaric) sulfate, octadecanoyl (stearic) sulfate, eicosanoyl (arachidic) sulfate, docosanoyl (behenic) sulfate, tetracosanoyl (lignoceric) sulfate, hexacosanoyl (cerotic) sulfate, heptacosanoyl (carboceric) sulfate, octacosanoyl (montanic) sulfate, triacontanoyl (melissic) sulfate, dotriacontanoyl (lacceroic) sulfate, tritriacontanoyl (ceromelissic) sulfate, tetratriacontanoyl (geddic) sulfate, and pentatriacontanoyl (ceroplastic) sufate.

23. The compound of claim 19, wherein the alkyl aryl sulfonate used to form the salt of methylene blue or a derivative of methylene blue is selected from the group consisting of dodecylbenzene sulfonate, tetradecanoylbenzene sulfonate, hexadecanoyl-benzene sulfonate, heptadecanoylbenzene sulfonate, octadecanoylbenzene sulfonate, eicosanoylbenzene sulfonate, docosanoylbenzene sulfonate, tetracosanoylbenzene sulfonate, hexacosanoylbenzene sulfonate, heptacosanoyl-benzene sulfonate, octasanoylbenzene sulfonate, triacontanoylbenzene sulfonate, dotriacontanoylbenzene sulfonate, triacontanoylbenzene sulfonate, tetratriacontanoylbenzene sulfonate, and pentatriacontanoylbenzene sulfonate.

24. The compound of formula 19, wherein the compound is methylene blue dodecylsulfate.

25. A method of making the pharmaceutical composition of 1, the method comprising converting methylene blue or a derivative of a methylene blue to a fatty acid, alkyl sulfate, aryl sulfate or alkyl arylsulfonate salt, and optionally, co-mixing or co-melting the fatty acid, alkyl sulfate, aryl sulfate or alkyl aryl sulfonate salt with a fatty acid.

26. The method of claim 25, wherein the salt of methylene blue is an alkyl sulfate salt.

27. The method of claim 26, wherein the alkyl sulfaate salt of methylene blue is methylene blue dodecylsulfate.

28. The method of claim 25, wherein the derivative of methylene blue has the chemical formula shown below: 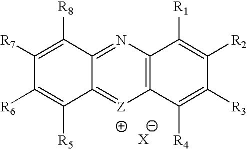
wherein R₁, R₂, R₄, R₅, R_{7 and R}₈ are independently selected from the group consisting of hydrogen, linear, branched or cyclic alkyl, aryl, substituted aryl, alkoxy, thioalkoxy, alkylamino, nitro, amino and halogen, R₃ and R₆ are independently selected from the group consisting of —OR₈, —NHR₉, and —NR₁₀R₁₁ and combinations thereof wherein R₈-R₁₁ is a linear, branched or cyclic hydrocarbon or R₁₀ and R₁₁ together with the nitrogen atom to which they are attached form an optionally substituted 5-, 6-, or 7-membered ring, wherein X is a counterion and wherein Z is either S or O and metabolites thereof.

29. The method of claim 25, wherein the derivative of methylene blue is selected from the group consisting of methyl methylene blue, dimethyl methylene blue, azure A, azure B, azure C, methylene green, new methylene blue, Taylor's Blue, toluidine Blue O, thionine, Nile blue, and metabolites thereof.

30. The method of calim 25, wherein the composition further comprises at least one other active agent selected from the group consisting of analgesics, antibiotics, antifungals, antivirals, anti-inflammatory drugs, antipyretics, nutritional agents, vitamins, and parasympathomimetics.

31. The method of claim 30, wherein the composition further comprises one or more vit amins selected fromthe group consisting of vitamins C, E, and B-complex.

32. The method of claim 25, wherein the composition is administered in a dosage form selected from the group consisting of tablets, soft gelatin capsules, hard shell capsules, suspensions, solutions, and suppositories.

33. The method of claim 25, wherein the composition is a controlled release formulation selected from the group consisting of immediate release, delayed release, extended release, pulsatile release, and combinations thereof.

34. The method of claim 25, wherein the fatty acid used to form the salt of methylene blue or a derivative of methylene blue is selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, triacontanoic (melissic) acid, dotriacontanoic (lacceroic) acid, tritriacontanoic (ceromelissic) acid, tetratriacontanoic (geddic) acid, and pentatriacontanoic (ceroplastic) acid.

35. The method of claim 25, wherein the dicarboxylic acid used to form the salt of methylene blue or a derivative of methylene blue is a dicarboxylic acid selected from the group consisting of succinic, glutaric, adipic, pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octendioic, and trans-3-octenedioic acid.

36. The method of claim 25, whrein the alkyl sulfate used to form the salt of methylene blue or a derivative of methylene blue is selected from the group consisting of decanoic (capric) sulfate, dodecyl (lauric) sulfate, tetradecanoyl (myristic) sulfate, hexadecanoyl (palmitic) sulfate, heptadecanoyl) (margaric) sulfate, octadecanoyl (stearic) sulfate, eicosanoyl (arachidic) sulfate, docosanoyl (behenic) sulfate, tetracosanoyl (lignoceric) sulfate, hexacosanoyl (cerotic) sulfate, heptcosanoyl (carboceric) sulfate, octacosanoyl (montanic) sulfate, triacontanoyl (melissic) sulfate, dotriacontanoyl (lacceroic0 sulfate, tritriacontanoyl (ceromelissic) sulfate, tetratriacontanoyl (geddic) sulfate, and petatriacontanoyl (ceroplastic) sulafate.

37. The method of claim 25, wherein the alkyl aryl sulfonate used to form the salt of methylene blue or a derivative of methylene blue is selected from the group consisting of dodecylbenzene sulfoante, tetradecanoylbenzene sulfonate, hexadecanoyl-benzene sulfonate, heptadecanoylbenzene sulfonate, octadecanoylbenzene sulfonate, eicosanoylbenzene sulfoante, docosanoylbenzene sulfonate, tetracosanoylbenzene sulfoante, hexacosanoylbenzene sulfonate, heptacosanoyl-benzene sulfonate, octacosanoylbenzene sulfonate, triacontanoylbenzene sulfonate, dotriacontanoylbenzene sulfonate, tritriacontanoylbenzene sulfonate, tetratriacontanoylbenzene sulfonate, and pentatriacontanoylbenzene sulfonate.

38. The method of claim 25, wherein the composition further comprises a fatty acid selected from the group consisting of butanoic (butyric) acid, pentanoic (valeric) acid, hexanoic (caproic) acid, octanoic (caprylic) acid, nonanoic (pelargonic) acid, decanoic (capric) acid, dodecanoic (lauric) acid, tetradecanoic (myristic) acid, hexadecanoic (palmitic) acid, heptadecanoic (margaric) acid, octadecanoic (stearic) acid, eicosanoic (arachidic) acid, docosanoic (behenic) acid, tetracosanoic (lignoceric) acid, hexacosanoic (cerotic) acid, heptacosanoic (carboceric) acid, octacosanoic (montanic) acid, tricontanoic (melissic) acid, dotriacontanoic (lacceroic) acid, tritriacontanoic (ceromelissic) acid, tetratriacontanoic (geddic) acid, and pentatriacontanoic (ceroplastic) acid.

39. The method of claim 25, wherein the composition further comprises a dicarboxylic acid selected from the group consisting of succinic, glutaric, adipic, pimelic, suberic, azelaic, sebacic, dodecanedioic, brassylic, thapsic, undecanedioic, tetradecanedioic, pentadecanedioic, hexadecanedioic, octadecanedioic, traumatic acid, itaconic (methylenesuccinic), trans-2-hexenedioic, trans-3-hexenedioic, cis-3-octenedioic, cis-4-octenedioic, and trans-3-octenedioic acid.

40. The method of claim 25, wherein the derivative of methylene blue is a metabolite of methylene blue which has the structure shown below:

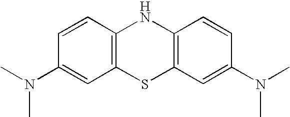

41. The method of claim 32, wherein the dosage form is a soft shell capsule.

42. The method of claim 41, further comprising methylene blue dissolved in the soft gelatin capsule shell to provide an immediate release portion.
